(19) United States
(12) Patent Application Publication
(10) Pub. No.: US 2022/0108805 A1
(43) Pub. Date: Apr. 7, 2022

(54) HEALTH MANAGEMENT APPARATUS AND HEALTH MANAGEMENT METHOD

(71) Applicant: SAMSUNG ELECTRONICS CO., LTD., Suwon-si (KR)

(72) Inventor: So Young LEE, Daejeon (KR)

(73) Assignee: SAMSUNG ELECTRONICS CO., LTD., Suwon-si (KR)

(21) Appl. No.: 17/211,283

(22) Filed: Mar. 24, 2021

(30) Foreign Application Priority Data
Oct. 7, 2020 (KR) 10-2020-0129270

(51) Int. Cl. G16H 50/30; G16H 50/20; A61B 5/145; A61B 5/00; A61B 5/1455

(52) U.S. Cl. CPC G16H 50/30; G16H 50/20; A61B 5/1455; A61B 5/7275; A61B 5/14532

(57) ABSTRACT

A health management apparatus for non-invasively managing a user's health is provided. The health management apparatus may include a spectrometer configured to obtain a spectrum from an object; a display; and a processor configured to predict a blood glucose variation based on the spectrum; calculate a glycemic variability index based on the blood glucose variation; generate a trend graph showing a change in the glycemic variability index; and control the display to display the trend graph via a user interface.

### CROSS-REFERENCE TO RELATED APPLICATION(S)

This application is based on and claims priority under 35 U.S.C. § 119 to Korean Patent Application No. 10-2020-0129270, filed on Oct. 7, 2020, in the Korean Intellectual Property Office, the disclosure of which is incorporated by reference herein in its entirety.

### BACKGROUND

### 1. Field

The disclosure relates to technology for managing a user's health by non-invasively calculating health indices such as a glycemic variability index.

### 2. Description of Related Art

Diabetes is a chronic disease that causes various complications and can be difficult to manage, such that people with diabetes are advised to check their blood glucose regularly to prevent complications. In particular, when insulin is administered to control blood glucose levels, the blood glucose levels have to be closely monitored to avoid hypoglycemia and control insulin dosage. An invasive method of finger pricking is generally used to measure blood glucose levels. However, while the invasive method may provide high reliability in measurement, it may cause pain and inconvenience as well as an increased risk of disease and infections due to the use of an injection. Recently, research has been conducted on methods to manage a user's health by non-invasively monitoring health indices, such as blood glucose, and the like, using a spectrometer without blood sampling.

### SUMMARY

According to an aspect of an example embodiment, a health management apparatus may include a spectrometer configured to obtain a spectrum from an object; a display; and a processor configured to predict a blood glucose variation based on the spectrum; calculate a glycemic variability index based on the blood glucose variation; generate a trend graph showing a change in the glycemic variability index; and control the display to display the trend graph via a user interface.

The processor is configured to predict the blood glucose variation based on the spectrum by using a blood glucose variation prediction model generated using at least one of classical least square (CLS), net analyte signal (NAS), and partial least squares (PLS).

The processor is configured to calculate the glycemic variability index including one or more of a moving average value, a standard deviation, a change in mean absolute glucose (MAG), a coefficient of variation, and a mean amplitude of glycemic excursion (MAGE), of the blood glucose variation.

The trend graph shows the change comprises at least one of a first visualization object visually representing positions of glycemic variability indices at predefined times and a second visualization object connecting the positions of the glycemic variability indices at the predefined times.

The processor is further configured to determine whether glycemic variability indices between two time points show a rising trend or a falling trend; and based on determining whether the glycemic variability indices between the two time points show a rising trend or a falling trend, vary at least one of a thickness, a color, and a type of the second visualization object connecting the positions of the glycemic variability indices at the two time points.

The processor is further configured to determine a diabetes risk level by comparing a trend of a change in glycemic variability indices between two time points with a predetermined threshold value; and based on determining the diabetes risk level, vary at least one of a thickness, a color, and a type of the second visualization object connecting the positions of the glycemic variability indices at the two time points.

The processor is further configured to detect a rising interval, in which the glycemic variability index continuously rises, or a falling interval in which the glycemic variability index continuously falls; and generate a third visualization object indicating a rising trend of the rising interval or a falling trend of the falling interval.

The processor is further configured to, based on a size of the rising interval or the falling interval, determine a size of the third visualization object.

The processor is further configured to, in response to the glycemic variability index being less than a first threshold value, determine that the glycemic variability index decreases; in response to the glycemic variability index being less than or equal to the first threshold value and being greater than or equal to a second threshold value, determine that the glycemic variability index is maintained; in response to the glycemic variability index exceeding the second threshold value, determine that the glycemic variability index increases; and generate a fourth visualization object showing that the glycemic variability index increases, is maintained, or decreases.

The processor is further configured to control the display to move a time axis of the trend graph, enlarge a predetermined interval, and select a predetermined time based on a user input received via the user interface.

The processor is further configured to predict a diabetes risk level by comparing a slope of a change in the glycemic variability index with a predetermined threshold value; and control the display to display guide information according to the diabetes risk level.

The processor is further configured to, predict a diabetes complication risk level by comparing an amplitude of the glycemic variability index during a predetermined period of time with a predetermined threshold value; and control the display to display guide information according to the diabetes complication risk level.

The processor is further configured to control the display to display at least one of the blood glucose variation, the glycemic variability index, and visualization reference information.

According to an aspect of an example embodiment, a health management method may include obtaining a spectrum from an object; predicting a blood glucose variation based on the spectrum; calculating a glycemic variability index based on the blood glucose variation; generating a trend graph showing a change in the glycemic variability index; and displaying the trend graph via a user interface.

The trend graph comprises at least one of a first visualization object visually representing positions of glycemic variability indices at predefined times, and a second visualization object connecting the positions of the glycemic variability indices at the predefined times.

The method may include determining whether glycemic variability indices between two time points show a rising trend or a falling trend; and based on determining whether the glycemic variability indices between the two time points show a rising trend or a falling trend, varying at least one of a thickness, a color, and a type of the second visualization object connecting the positions of the glycemic variability indices at the two time points.

The method may include determining a diabetes risk level by comparing a trend of a change in glycemic variability indices between two time points with a predetermined threshold value; and based on determining the diabetes risk level, varying at least one of a thickness, a color, and a type of the second visualization object connecting the positions of the glycemic variability indices at the two time points.

The method may include detecting a rising interval, in which the glycemic variability index continuously rises, or a falling interval in which the glycemic variability index continuously falls; and generating a third visualization object indicating a rising trend of the rising interval or a falling trend of the falling interval.

The method may include in response to the glycemic variability index being less than a first threshold value, determining that the glycemic variability index decreases; in response to the glycemic variability index being less than or equal to the first threshold value and being greater than or equal to a second threshold value, determining that the glycemic variability index is maintained; in response to the glycemic variability index exceeding the second threshold value, determining that the glycemic variability index increases; and generating a fourth visualization object showing that the glycemic variability index increases, is maintained, or decreases.

The method may include predicting a diabetes risk level by comparing a slope of a change in the glycemic variability index with a predetermined threshold value; and displaying guide information according to the diabetes risk level.

The method may include predicting a diabetes complication risk level by comparing an amplitude of the glycemic variability index during a predetermined period of time with a predetermined threshold value; and displaying guide information according to the diabetes complication risk level.

Additional aspects will be set forth in part in the description which follows and, in part, will be apparent from the description, or may be learned by practice of the presented embodiments.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a block diagram illustrating a health management apparatus according to an example embodiment;

FIG. 2 is a schematic diagram illustrating a structure of a spectrometer according to an example embodiment;

FIG. 3 is a block diagram illustrating a processor according to an example embodiment;

FIGS. 4A to 4E are diagrams illustrating examples of visualizing a glycemic variability index according to an example embodiment;

FIGS. 5A to 5C are diagrams illustrating examples of visualization according to a user's action performed on a user interface according to an example embodiment;

FIGS. 6A and 6B are diagrams illustrating examples of health guide information based on monitored glycemic variability according to an example embodiment;

FIG. 7 is a flowchart illustrating a health management method according to an example embodiment;

FIG. 8 is a diagram illustrating a wearable device as an example of a health management apparatus according to an example embodiment; and

FIG. 9 is a diagram illustrating a smart device as another example of a health management apparatus according to an example embodiment.

Throughout the drawings and the detailed description, unless otherwise described, the same drawing reference numerals will be understood to refer to the same elements, features, and structures. The relative size and depiction of these elements, features, and structures may be exaggerated for clarity, illustration, and convenience.

### DETAILED DESCRIPTION

Details of example embodiments are included in the following detailed description and drawings. Advantages and features of the present disclosure, and a method of achieving the same will be more clearly understood from the following embodiments described in detail with reference to the accompanying drawings. Throughout the drawings and the detailed description, unless otherwise described, the same drawing reference numerals will be understood to refer to the same elements, features, and structures.

It will be understood that, although the terms “first,” “second,” etc. may be used herein to describe various elements, these elements should not be limited by these terms. These terms are only used to distinguish one element from another. Any references to the singular form of a term may include the plural form of the term unless expressly stated otherwise. In addition, unless explicitly described to the contrary, an expression such as “comprising” or “including” will be understood to imply the inclusion of the stated elements but not the exclusion of any other elements. Also, the terms, such as “unit,” “module,” etc., should be understood as a unit that performs at least one function or operation and that may be embodied as hardware, software, or a combination thereof.

Hereinafter, embodiments of an apparatus and method for estimating an analyte concentration will be described below with reference to the accompanying drawings.

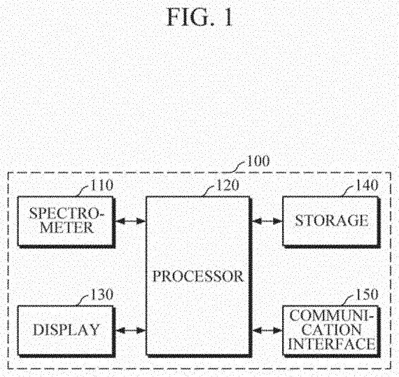

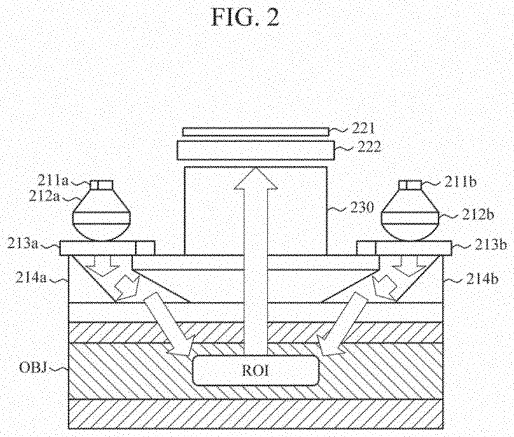

FIG. 1 is a block diagram illustrating a health management apparatus according to an embodiment of the present disclosure. FIG. 2 is a schematic diagram illustrating a structure of a spectrometer.

Referring to FIG. 1, the health management apparatus 100 includes a spectrometer 110, a processor 120, a display 130, a storage 140, and a communication interface 150. Various components of the health management apparatus 100 may be mounted in a single hardware device. In this case, the communication interface 150 may be omitted according to the size and/or purpose of the health management apparatus 100. Further, the health management apparatus 100 may be provided separately as two or more hardware devices to perform in conjunction with each other a function of managing a user's health. For example, the spectrometer 110 and the communication interface 150 may be provided in a wearable device, such as earphones, and the like, and the processor 120, the display 130, and the storage 140 may be provided in another hardware device, such as a smartphone.

The spectrometer 110 may measure a spectrum from an object. The spectrometer 110 may include one or more light sources configured to emit light onto the object periodically or non-periodically under the control of the processor 120, and a detector configured to obtain a spectrum by detecting light that reacts with the object by being scattered by, reflected from, or transmitted into the object. The object may be a body part and may be, for example, an area where veins or capillaries are located or an area adjacent to the radial artery, and may be a peripheral part of the body, such as fingers, toes, earlobes, etc., where blood vessels are densely distributed.

The light source may be provided as a light emitting diode (LED), a laser diode (LD), a phosphor, and the like. The light source may emit light in a wavelength range of 1500 nm to 1850 nm or in a wavelength range of 2000 nm to 2400 nm, so as to measure spectra by using, for example, near-infrared spectroscopy (NIRS). However, the light source is not limited thereto, and may emit single laser light, mid-infrared light, and the like. The detector may include a single photo diode or a photo diode array to detect light from the object and to convert the detected light into an electrical signal. However, the detector is not limited thereto, and may also be formed as a photo transistor (PTr), an image sensor (e.g., a complementary metal-oxide-semiconductor (CMOS) image sensor), and the like.

Referring to FIG. 2, the spectrometer 110 according to an embodiment may include a detector 221, disposed at the center thereof, and one or more light sources 211a and 211b disposed around the detector 221 in various shapes. For example, the plurality of light sources 211a and 211b may be disposed in a circular or rectangular shape on the outside of the detector 221. The light sources 211a and 211b may emit light of different wavelengths. The light sources 221a and 211b may be driven sequentially or simultaneously in a time-division manner under the control of the processor 120 in a predetermined direction (e.g., clockwise direction) or in order of wavelength. However, the light sources 211a and 211b are not limited thereto, and may be divided into groups of light sources to be driven sequentially or simultaneously in groups.

In addition, the spectrometer 110 may further include lenses 212a and 212b for focusing light, emitted by the light sources 211a and 211b, toward the object. The spectrometer 110 may further include filters 213a and 213b for filtering the light into a predetermined wavelength. The spectrometer 110 may further include external reflectors 214a and 214b for redirecting the light, having passed through the filters 213a and 213b, toward a region of interest (ROI) of the object OBJ. In this case, the external reflectors 214a and 214b may be formed as optical mirrors. Further, the spectrometer 110 may include an internal reflector 230 configured to form a light path so that light, scattered or reflected from or transmitted into the surface of the object OBJ or an internal portion, particularly a region of interest (ROI), of the object OBJ, may be directed toward the detector 221. The internal reflector 230 may be provided as a partition wall having an external part for shielding light and an internal part formed with an optical mirror to direct light toward the detector 221. In addition, the detector 221 may further include a filter 222, formed on a front surface thereof, for passing light of a predetermined wavelength after reacting with the object.

The processor 120 may be electrically connected to the spectrometer 110, directly or through wireless communication, to control driving of the spectrometer 110. Based on receiving spectrum data of the object from the spectrometer 110, the processor 120 may provide health information by monitoring variability of various health-related substances of the object. In this case, the object substance may include blood glucose, cholesterol, triglyceride, protein, uric acid, carotenoid, alcohol, water, and the like, but is not limited thereto. For convenience of explanation, the following description will be given using blood glucose as an example.

The processor 120 may predict a blood glucose variation by using spectrum data, may calculate a glycemic variability index, and may visualize the calculated glycemic variability index to provide the visualized index for a user. For example, by rendering related information based on the glycemic variability index, so that a user may visually identify glycemic variability or health-related information, the processor 120 may generate the information as visualized images. Based on calculating a change in blood glucose concentration at a current time, the processor 120 may estimate a blood glucose level by using a pre-defined blood glucose concentration estimation model. In this case, the blood glucose concentration estimation model may be expressed as linear or non-linear functions for estimating blood glucose by using a reference blood glucose level obtained at a calibration time.

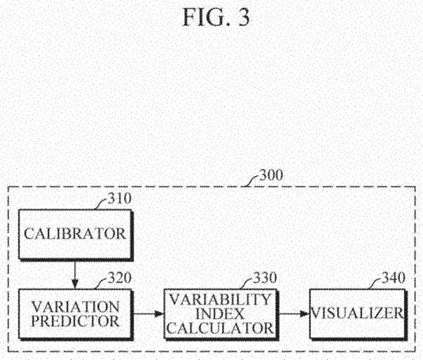

FIG. 3 is a block diagram illustrating a processor according to an embodiment of the present disclosure.

Referring to FIG. 3, the processor 300 according to an embodiment of the present disclosure includes a calibrator 310, a variation predictor 320, a variability index calculator 330, and a visualizer 340.

The calibrator 310 may calibrate a blood glucose variation prediction model in response to an input from a user or an external device, or if predetermined criteria are satisfied. In this case, the predetermined criteria may be set such that calibration is performed periodically at regular intervals or is performed non-periodically based on a prediction result of a blood glucose variation and the like.

The calibrator 310 may obtain, as training data, a spectrum obtained from a user and/or a background spectrum measured while a user is in a resting state. In addition, the calibrator 310 may generate and train a blood glucose variation prediction model by using the training data. For example, the blood glucose variation prediction model may be generated and trained by using the following Equation 1 based on Lambert-Beer's Law. In this case, the prediction model may be trained using methods such as a regression algorithm, net analyte signal (NAS), and/or artificial intelligence, and the regression algorithm may include linear regression, non-linear regression, classical least square, partial least squares, neural network regression, support vector regression, decision tree based regression, Naive Bayes regression, and the like, but is not limited thereto.

S=BS+ε_{g}·L·Cₜ [Equation 1]

Herein, S denotes the spectrum measured from a user; and BS denotes the background spectrum measured in a resting state, and may include a signal of a skin component except a blood glucose component. In this case, the resting state may be a fasting state of a user. Further, ε_{g}·L·ΔCₜ denotes a blood glucose component signal, ε_{g} denotes a unit spectrum of blood glucose, and L denotes a light transfer path. In this case, the unit spectrum of blood glucose and the light transfer path are pre-input values; and ΔCₜ denotes a change in blood glucose concentration.

Based on the spectrometer 110 obtaining the spectrum at the current time from the object, the variation predictor 320 may predict a blood glucose variation at the current time compared to a previous time. In this case, the blood glucose variation may be predicted periodically (e.g., in units of several minutes, hours, days, weeks, months, etc.) or non-periodically (e.g., when a user requests). For example, the variation predictor 320 may predict a blood glucose variation compared to a previous time, by using the above Equation 1 and the following Equation 2.

ΔG=G(t+ΔT)−G(t) [Equation 2]

Herein, ΔG denotes a blood glucose variation between a first time t and a second time t+ΔT; G (t) denotes a blood glucose level at the time t; and G (t+ΔT) denotes a blood glucose level at the second time t+ΔT. Here, blood glucose levels at each time may be defined as a value obtained by adding an offset (e.g., reference blood pressure measured in a fasting state) to a change ΔCₜ in blood glucose concentration obtained at each time by using the above Equation 1. Assuming that the reference pressure is the same at the first time t and the second time t+ΔT, the blood glucose variation ΔG between the first time t and the second time t+ΔT may be obtained by subtracting the change in blood glucose concentration at the first time t from the change in blood glucose concentration at the second time t+ΔT, which are obtained using the above Equation 1. In this case, the change in blood glucose concentration may be calculated without directly measuring a blood glucose value, such that a process of invasively obtaining a reference blood pressure at the calibration time may be omitted.

The variability index calculator 330 may calculate a glycemic variability index by using accumulated data of blood glucose variations. In this case, the glycemic variability index may include, but is not limited to, a moving average value, a standard deviation, a change in mean absolute glucose (MAG), a coefficient of variation, a mean amplitude of glycemic excursion (MAGE), and the like, of blood glucose variations. For example, the variability index calculator 330 may obtain a moving average value by taking a moving average of accumulated data of blood glucose variations, stored in the storage 140, in units of predetermined periods, such as 10 minutes, 10 hours, a week, and the like. Alternatively, the variability index calculator 330 may calculate an index, such as a standard deviation, a change in MAG, and the like, in units of weeks/months by using blood glucose variation data accumulated during a predetermined period of time, such as a period of one week/one month. However, the calculation of the index is not limited thereto, and the period may be set by considering user characteristics. For example, the predetermined period or the period of calculating the index may be set to a relatively short period for diabetic patients; and for normal healthy users, the predetermined period or the period of calculating the index may be set to a relatively long period.

Based on the variability index calculator 330 calculating the glycemic variability index, the visualizer 340 may visualize the glycemic variability index so that a user may monitor the glycemic variability easily and visually. For example, the visualizer 340 may generate a visualized image by performing two-dimensional (2D)/three-dimensional (3D) rendering using the glycemic variability index, the data used for calculating the glycemic variability index, and/or health guide information based on the monitored glycemic variability. In this case, the visualized image may include a trend graph showing a change in glycemic variability index; and the trend graph showing a change in glycemic variability index may include various visualization objects, so that a user may easily and visually monitor their health condition, such as glycemic variability and/or diabetes risk, diabetic complication risk, and the like. In addition, the visualizer 340 may process, in real time, a user's request by responding to a user's input by interaction with a user interface, and may visualize the data by rendering the processed data.

Referring to FIG. 1, the display 130 may provide a user interface for a user, and may display a visualization result of the processor 120, such as the visualized image, on the user interface. The display 130 may include touch circuitry adapted to detect a touch and/or sensor circuitry, such as a pressure sensor, etc., configured to measure the intensity of force incurred by the touch.

The user interface may provide various functions for interaction with the user and the processor 120. Based on the processor 120 processing visualization of the glycemic variability index, the user interface may display a visualization result in response to the visualization by the processor 120, may transmit a user command to the processor 120 in response to a user's various actions performed on the user interface, and may display a visualization result processed by the processor 120 according to the user command. For example, the user interface may output various visualization objects for receiving the user command. The user may select the visualization objects by using actions such as a click, a double click, and a drag-and-drop, and/or a pre-defined gesture action; or may enter a pre-defined command, such as moving a time axis, selecting a specific time, enlarging a specific interval, and the like.

The storage 140 may store data generated, received, processed, or used by various components of the health management apparatus 100, such as the spectrometer 110, the processor 120, the communication interface 150, and the like. For example, the storage 140 may store spectrum data obtained by the spectrometer 110, and may manage the accumulated spectrum data. Alternatively, the storage 140 may store a predicted blood glucose variation value processed by the processor 120, the calculated glycemic variability index, and/or the data such as the generated visualized imaged and the like, and may accumulate and manage the data including the predicted blood glucose variation value, the glycemic variability index, and the like. Further, the storage 140 may store light source driving conditions, such as current intensity, duration, and driving sequence of light sources, for controlling the spectrometer 110, and information, such as a blood glucose variation prediction model, calibration conditions, and the like, for visualization processed by the processor 120. In addition, the storage 140 may store data received from an external device through the communication interface 150.

The storage 140 may include a storage medium of a flash type memory, a hard disk type memory, a multimedia card micro type memory, a card type memory (e.g., a secure digital (SD) memory, an extreme digital (XD) memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

The communication interface 150 may transmit the data stored in the storage 140 to an external device by using wired or wireless communication techniques under the control of the processor 120, and may receive a variety of information from the external device and store the information in the storage 140. In this case, the external device may include an apparatus for measuring a bio-signal in an invasive/minimally invasive manner, and an information processing device such as a smartphone, a tablet personal computer (PC), a desktop computer, a laptop computer, and the like.

In this case, examples of the communication techniques may include, but are not limited to, Bluetooth communication, Bluetooth Low Energy (BLE) communication, Near Field Communication (NFC), wireless local area network (WLAN) communication, Zigbee communication, Infrared Data Association (IrDA) communication, wireless fidelity (Wi-Fi) Direct (WFD) communication, Ultra-Wideband (UWB) communication, Ant+ communication, Wi-Fi communication, Radio Frequency Identification (RFID) communication, 3G communication, 4G communication, 5G communication, and the like.

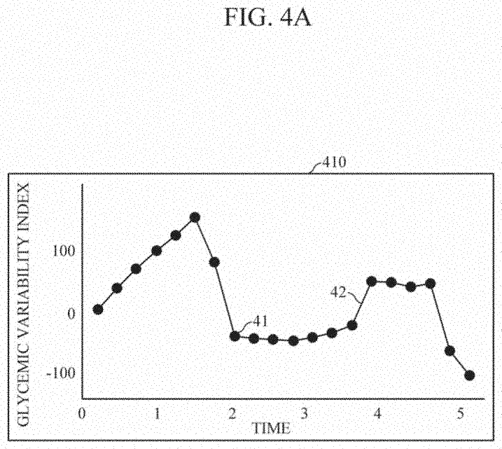

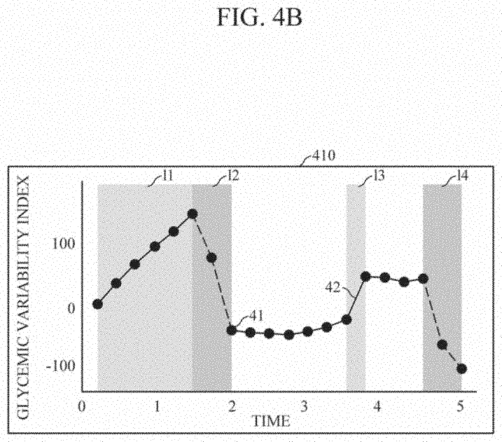

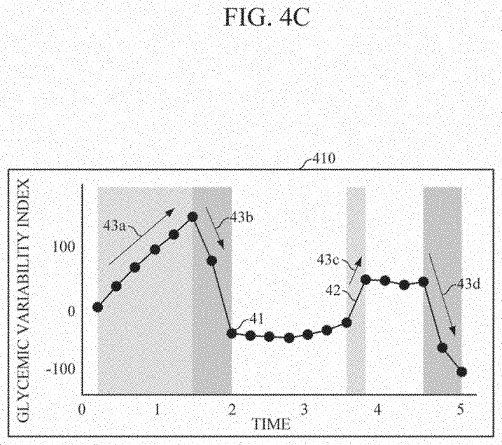

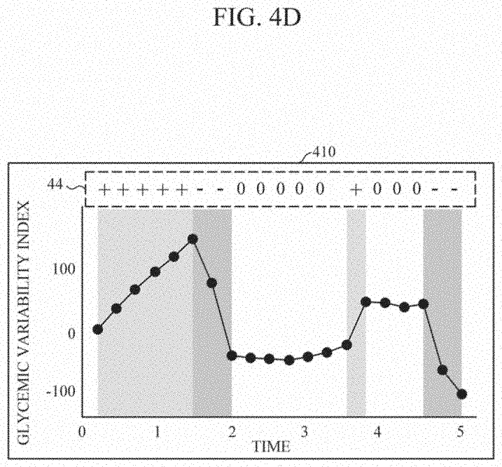

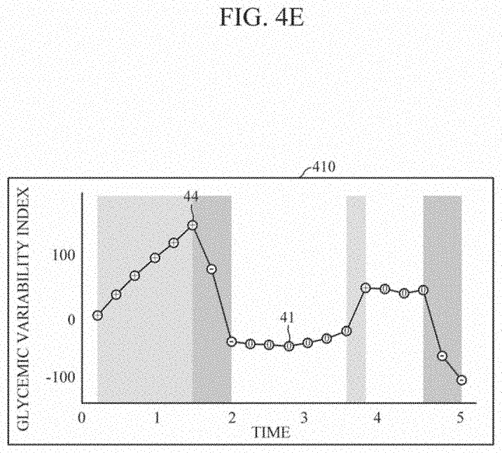

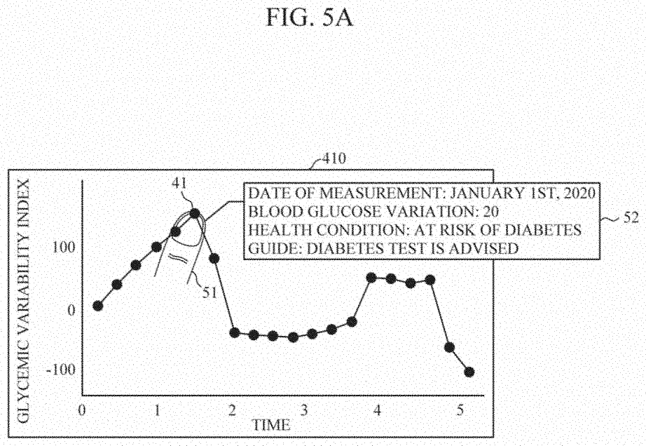

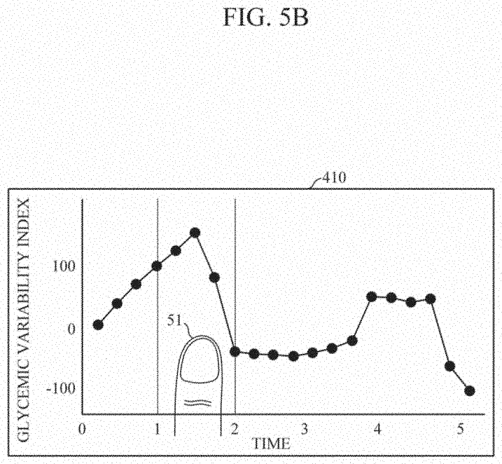

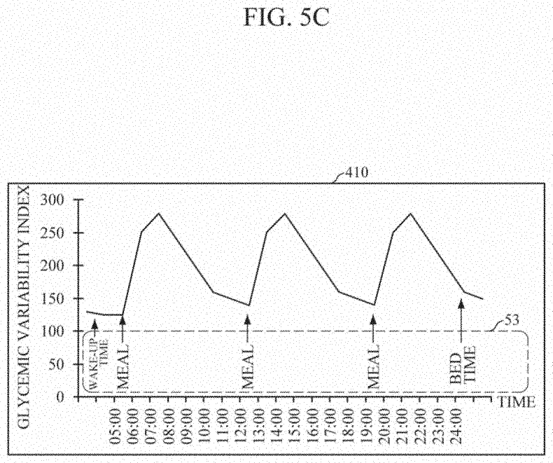

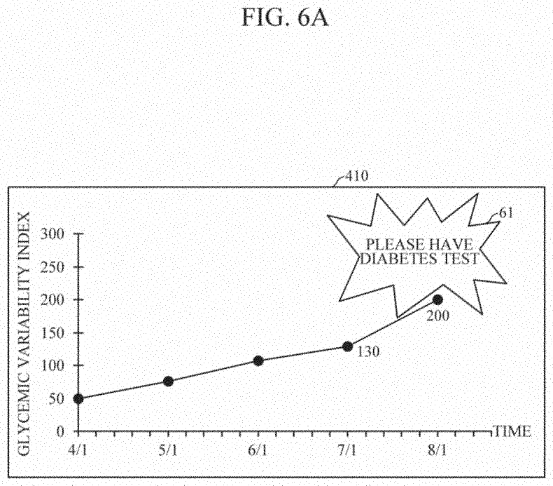

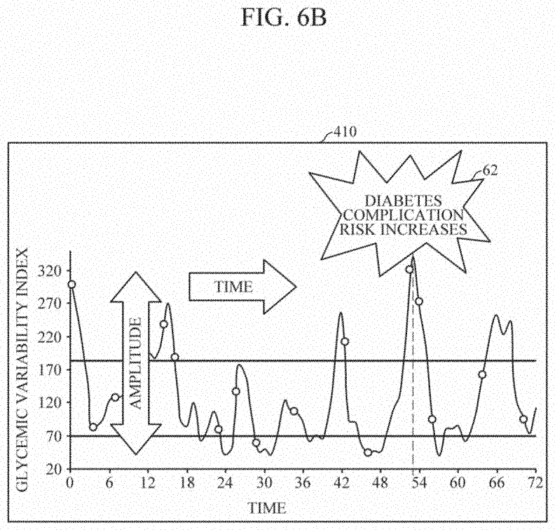

FIGS. 4A to 4E are diagrams illustrating examples of visualizing a glycemic variability index. FIGS. 5A to 5C are diagrams illustrating examples of visualization according to a user's action performed on a user interface. FIGS. 6A and 6B are diagrams illustrating examples of health guide information based on monitored glycemic variability.

Various embodiments of visualization for monitoring glycemic variability performed by the processor 120 and the display 130 will be described below with reference to FIGS. 1 and 4A to 6B.

FIGS. 4A to 4E are trend graphs showing a change in glycemic variability index output on a user interface 410 of the display 130. The X axis represents a time index indicating a default value stored in the storage 140 or a time unit according to a user's setting. For example, each number on the X axis may indicate values such as time, day, month, and the like. The Y axis represents a glycemic variability index and may be, for example, a moving average value of blood glucose variations.

The processor 120 may generate a trend graph showing a change in glycemic variability index in various forms such as a line graph, a bar graph, and the like. The processor 120 may generate the trend graph showing the change by including various visualization objects, so that a user may visually identify a variety of information represented by the trend graph showing the change in glycemic variability index. Referring to FIG. 4A, the processor 120 may provide a first visualization object 41 on the position of each glycemic variability index, so that a user may easily identify the position of glycemic variability index at each time. In this case, the first visualization object 41 may have a circular shape, a square shape, a triangular shape, and the like, with no limitations on its shape, texture, color, size, and the like. In addition, the processor 120 may further include a second visualization object 42 connecting the positions of glycemic variability indices at each time. The second visualization object 42 may be a solid line, a dotted line, a double solid line, a double dotted line, and the like, with no limitations on the type, thickness, color, and the like of the lines.

The processor 120 may determine whether a glycemic variability index shows a rising trend or a falling trend over time, and may visualize the determination result. If a change with time in glycemic variability index at a second time compared to a first time, such as a value obtained by subtracting the glycemic variability index at the first time from the glycemic variability index at the second time, is greater than a first threshold value (positive value), the processor 120 may determine that a glycemic variability index “increases.” Alternatively, if the value is greater than or equal to a second threshold value (negative value) and is less than or equal to the first threshold value, the processor 120 may determine that the glycemic variability index “is maintained.” Alternatively, if the value is less than the second threshold value, the processor 120 may determine that the glycemic variability index “decreases.”

Referring to FIG. 4B, based on the rise and fall of the glycemic variability index, the processor 120 may vary the thickness, color, and type of the second visualization object 42 connecting glycemic variability indices at two time points. For example, as illustrated in FIG. 4B, lines of the second visualization object 42 connecting the two time points in rising intervals 11 and 13 may be shown by a solid line. Here, in a maintaining interval, which does not include the rising intervals 11 and 13 and falling intervals 12 and 14, the second visualization object 42 is shown by a solid line. In contrast, the solid line in the rising intervals 11 and 13 may be shown by a thick solid line, a double solid line, a red solid line, and the like, and the line in the maintaining interval may be shown by a solid line, a gray solid line, and the like, so that the lines in the respective intervals may be distinguishable from each other. In addition, as illustrated in FIG. 4B, the rising intervals 11 and 13 and the falling intervals 12 and 14 may be shown in different colors so that the respective intervals may be distinguishable from each other.

However, the processor 120 is not limited thereto, and based on whether the glycemic variability index rises or falls, the processor 120 may vary the shape, color, texture, and the like of the first visualization object 41. For example, if the glycemic variability index shows a rising trend at the current time compared to a previous time, the processor 120 may display the first visualization object 41 in red and/or in a square shape; if the glycemic variability index shows a falling trend, the processor 120 may display the first visualization object 41 in blue and/or in a triangular shape; and if the glycemic variability index is in a maintaining interval, the processor 120 may display the first visualization object 41 in black and/or in a circular shape.

Referring to FIG. 4C, the processor 120 may determine an interval in which the glycemic variability index continuously rises or falls, and may generate third visualization objects 43a, 43b, 43c, and 43d indicating a rising trend or a falling trend of each interval. As illustrated in FIG. 4C, the third visualization object may be an arrow. In this case, based on a size of the continuously rising or falling interval, the processor 120 may determine a size of the third visualization object and may generate the third visualization object based on the determined size.

Referring to FIGS. 4D and 4E, the processor 120 may determine whether the glycemic variability index, such as a moving average, at a specific time “increases,” “is maintained,” or “decreases” and may generate a fourth visualization object 44 by visualizing the determination result. For example, if a value, obtained by subtracting a moving average at a previous time (e.g., a preceding time) from a moving average at a current time, or a statistical value (e.g., a mean value) of moving averages during a previous predetermined period of time including the current time, is less than a first threshold value, the processor 120 may determine that the glycemic variability index “falls.” Alternatively, if the value is greater than or equal to a second threshold value and is less than or equal to the first threshold value, the processor 120 may determine that the glycemic variability index “is maintained.” Alternatively, if the value exceeds the second threshold value, the processor 120 may determine that the glycemic variability index “increases.” As illustrated in FIGS. 4D and 4E, if the value “falls,” the fourth visualization object may be a negative sign “−,” if the value “is maintained,” the fourth visualization object may be “0,” and if the value “increases,” the fourth visualization object may be a positive sign “+.” However, the fourth visualization object is not limited thereto, and the processor 120 may visualize the values using various shapes and/or colors so that the values may be distinguishable by shape and/or color. As illustrated in FIG. 4D, the fourth visualization object 44 may be displayed in a predetermined area of the user interface 410, such as at positions corresponding to each time at the top of the trend graph of the change. Alternatively, as illustrated in FIG. 4E, the fourth visualization object 44 may be displayed on the first visualization object on the trend graph of the change.

FIGS. 5A to 5C are diagrams explaining an example of processing a user command performed on the user interface 410.

Referring to FIGS. 5A to 5C, a user may check a trend of a blood glucose change on the user interface 410, and in order to further obtain desired information, the user may perform various actions such as selecting a specific time, enlarging a specific interval, moving a time axis (e.g., moving to a previous/subsequent interval in time intervals displayed on the trend graph of the current change), and the like, on the trend graph of the change. The user may perform various pre-defined actions (e.g., a click, a double click, a drag and drop, etc.) on the user interface 410 using various input means, such as a mouse, a touch pen, a finger, and the like, or may enter a user command by performing various pre-defined gesture actions. The user interface 410 may interact in real time with the processor 120 to respond in real time to the actions performed by the user. For example, the processor 120 may control the display 130 to move a time axis of the trend graph, enlarge a predetermined interval, and select a predetermined time based on a user input received via the user interface 410.

For example, as illustrated in FIG. 5A, when a user clicks on the first visualization object 41 at a specific time with a finger 51, detailed information 52 corresponding to the selected time may be displayed in a specific area of the user interface 410. Alternatively, when a user double clicks on a specific interval with the finger 51 as illustrated in FIG. 5B, the trend graph showing a change in glycemic variability index corresponding to the selected interval may be displayed in detail on the user interface 410 as illustrated in FIG. 5C. In this case, as illustrated in FIG. 5C, additional information 53 according to a blood glucose change of the selected interval may be further displayed on the trend graph of the change. In addition, although not illustrated herein, when a user moves a finger to the right while touching a specific coordinate position, a trend graph of the change in subsequent time intervals (e.g., 6-10 in the time index) may be displayed. However, FIGS. 5A to 5C are merely examples, and various other actions may be performed.

FIGS. 6A and 6B are diagrams illustrating examples of displaying health guide information based on a change in glycemic variability index.

Referring to FIG. 6A, for normal users, the processor 120 may predict a diabetes risk level based on the glycemic variability index, and may visualize a visualization object 61 displaying guide information according to a diabetes risk level based on the prediction result. For example, assuming that a threshold value is 50, a glycemic variability index at a time of 7/1 is 130, and a glycemic variability index at a time of 8/1 is 200, such that a change in glycemic variability index between the two time points is 70, which exceeds 50, and as a result, the processor 120 may determine that diabetes risk increases at the 8/1 time. In addition, as illustrated in FIG. 6A, the processor 120 may display the visualization object 61 displaying guide information such as “please have diabetes test.” Alternatively, the processor 120 may divide diabetes risk into diabetes, prediabetes, and normal levels; and if a change in glycemic variability index exceeds a first threshold value, the processor 120 may classify the diabetes risk as a prediabetes level, and if a change in glycemic variability index exceeds a second threshold value, the processor 120 may classify the diabetes risk as diabetes level, and may display guide information according to a corresponding level.

Referring to FIG. 6B, for diabetic patients, the processor 120 may monitor a glycemic variability index and may determine a diabetic complication risk level, such as a probability of abnormalities in the cardiovascular system, and may visualize guide information based on the determination result. For example, if an amplitude of a glycemic variability index in a predetermined interval, such as a difference between a maximum value and a minimum value, exceeds a predetermined threshold value, the processor 120 may determine that the diabetic complication risk increases. In this case, if a number of times that the amplitude exceeds the predetermined threshold value is greater than or equal to a predetermined number of times, the processor 120 may determine that the diabetic complication risk increases. Alternatively, the processor 120 may calculate an amplitude of a glycemic variability index in units of predetermined periods of time, and if the calculated amplitude gradually increases with time, the processor 120 may determine that the diabetic complication risk increases. As illustrated in FIGS. 6A and 6B, if the amplitude exceeds the threshold value around time index 54, the processor 120 may display a visualization object 62 including guide information such as “diabetic complication risk increases.” However, FIGS. 6A and 6B are merely examples.

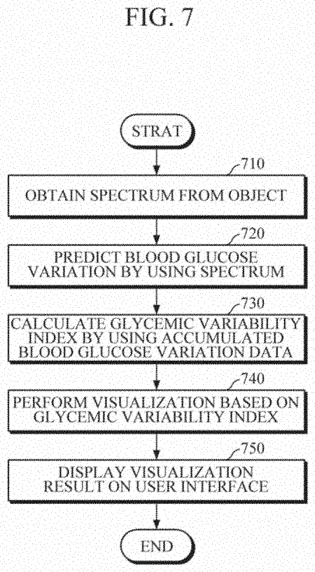

FIG. 7 is a flowchart illustrating a health management method according to an embodiment of the present disclosure.

The method of FIG. 7 is an example of a health management method performed by the health management apparatus 100 of FIG. 1, which is described above in detail, and thus will be briefly described below.

The health management apparatus 100 may obtain a spectrum, reacted with an object by being scattered by, reflected from, or transmitted into the object, by using a spectrometer in operation 710.

The health management apparatus 100 may predict a blood glucose variation by using the obtained spectrum in operation 720. The health management apparatus 100 may periodically or non-periodically predict the blood glucose variation by using a pre-defined blood glucose variation prediction model based on Equations 1 and 2 shown elsewhere herein.

The health management apparatus 100 may calculate a glycemic variability index by using accumulated blood glucose variation data in operation 730. In this case, the glycemic variability index may include a moving average value, a standard deviation, a change in mean absolute glucose (MAG), a coefficient of variation, a mean amplitude of glycemic excursion (MAGE), and the like, of blood glucose variations. The glycemic variability index may be calculated in units of predetermined periods, such as minutes, hours, days, weeks, months, and the like.

The health management apparatus 100 may perform visualization based on the calculated glycemic variability index in operation 740. For example, the health management apparatus 100 may generate a visualized image by using the glycemic variability index, data for calculating the glycemic variability index, health guide information, and the like. In this case, the visualized image may include a trend graph showing a change in glycemic variability index, and may include various visualization objects so that a user may easily and visually monitor health conditions, such as glycemic variability and/or diabetes risk, diabetic complication risk, and the like.

The health management apparatus 100 may display a visualization result on a user interface in operation 750, and may interact with a user to process a user's request in real time in response to a user's input through the user interface.

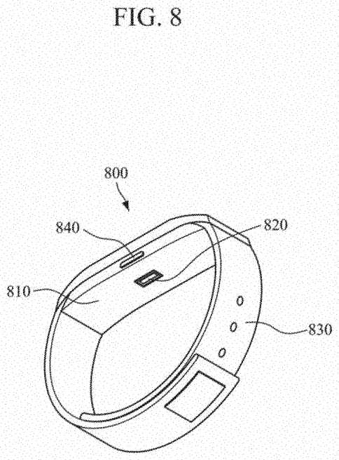

FIG. 8 is a diagram illustrating a wearable device as an example of a health management apparatus. FIG. 8 illustrates a wearable device 800 implemented as a smart watch worn on a user.

The wearable device 800 includes a main body 810 and a strap 830. The main body 810 may be worn on a user's wrist with the strap 830. The main body 810 may include the health management apparatus 100 of the wearable device 800 and various modules performing various other functions. A battery may be embedded in the main body 810 or the strap 830 to supply power to the various modules of the wearable device 800. The strap 830 may be flexible so as to be wrapped around a user's wrist. The strap 830 may include a first strap and a second strap which are separated from each other. Respective ends of the first strap and the second strap are connected to the main body 810, and the other ends thereof may be connected to each other via a connecting means. In this case, the connecting means may be formed as magnetic connection, Velcro connection, pin connection, and the like, but is not limited thereto. Further, the strap 830 is not limited thereto, and may be integrally formed as a non-detachable band.

The main body 810 may include a spectrometer 820 formed on one surface thereof, and a processor is disposed inside the main body 810 to be electrically connected to the spectrometer 820. The processor may monitor glycemic variability by using a spectrum obtained by the spectrometer 820, and/or estimate blood glucose levels, and may visualize information related to glycemic variability and/or blood glucose-related information.

Further, the main body 810 may include a storage for storing data generated and/or processed by the wearable device 800, and a communication interface for communicating with an external device to transmit and receive data.

The main body 810 may include a manipulator 840 which is provided on one side surface of the main body 810, and receives a user's control command and transmits the received control command to the processor. The manipulator 840 may have a power button to input a command to turn on/off the wearable device 800.

Further, a display for outputting information to a user may be mounted on a front surface of the main body 810. The display may have a touch screen for receiving touch input. The display may interact with the processor and the user to output a visualization result of the processor and to receive the user's command and transmit the command to the processor.

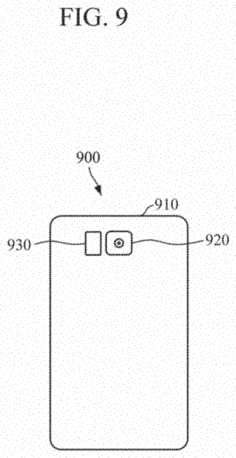

FIG. 9 is a diagram illustrating a smart device as another example of a health management apparatus.

The smart device 900 may include a main body 910 and a spectrometer 930 mounted on a rear surface of the main body 910. A processor may be disposed inside the main body 910 of the smart device 900, and the processor may be electrically connected to the spectrometer 930 to monitor glycemic variability by using a spectrum obtained by the spectrometer 820, and may estimate blood glucose levels.

A camera module 920 may be disposed on a rear surface of the main body 910. The camera module 920 may capture still images or moving images. The camera module 920 may include a lens assembly having one or more lenses, image sensors, image signal processors, and/or flashes.

A display may be mounted on a front surface of the main body 910 of the smart device 900. The display may visually output various data generated and/or processed by the mobile device 900. The display may interact with the user and the processor to output a visualization result of the processor and to transmit a user command to the processor by using the visualization result.

The example embodiments of the present disclosure may be implemented by computer-readable code written on a non-transitory computer-readable medium. The non-transitory computer-readable medium may be any type of recording device in which data is stored in a computer-readable manner.

Examples of the computer-readable medium include a ROM, a RAM, a CD-ROM, a magnetic tape, a floppy disc, an optical data storage, and a carrier wave (e.g., data transmission through the Internet). The computer-readable recording medium can be distributed over a plurality of computer systems connected to a network so that a computer-readable code is written thereto and executed therefrom in a decentralized manner. Functional programs, code, and code segments for implementing example embodiments of the present disclosure can be readily deduced by programmers of ordinary skill in the art to which the present disclosure pertains.

The present disclosure has been described herein with regard to example embodiments. However, it will be obvious to those skilled in the art that various changes and modifications can be made without changing technical concepts of the present disclosure. Thus, it is clear that the above-described embodiments are illustrative in all aspects and are not intended to limit the present disclosure.

What is claimed is:

1. A health management apparatus, comprising:
a spectrometer configured to obtain a spectrum from an object;
a display; and
a processor configured to:
predict a blood glucose variation based on the spectrum;
calculate a glycemic variability index based on the blood glucose variation;
generate a trend graph showing a change in the glycemic variability index; and
control the display to display the trend graph via a user interface.

2. The health management apparatus of claim 1, wherein the processor is configured to predict the blood glucose variation based on the spectrum by using a blood glucose variation prediction model generated using at least one of classical least square (CLS), net analyte signal (NAS), and partial least squares (PLS).

3. The health management apparatus of claim 1, wherein the processor is configured to calculate the glycemic variability index comprising one or more of a moving average value, a standard deviation, a change in mean absolute glucose (MAG), a coefficient of variation, and a mean amplitude of glycemic excursion (MAGE), of the blood glucose variation.

4. The health management apparatus of claim 1, wherein the trend graph showing the change comprises at least one of a first visualization object visually representing positions of glycemic variability indices at predefined times and a second visualization object connecting the positions of the glycemic variability indices at the predefined times.

5. The health management apparatus of claim 4, wherein the processor is further configured to:
determine whether glycemic variability indices between two time points show a rising trend or a falling trend; and
based on determining whether the glycemic variability indices between the two time points show a rising trend or a falling trend, vary at least one of a thickness, a color, and a type of the second visualization object connecting the positions of the glycemic variability indices at the two time points.

6. The health management apparatus of claim 4, wherein the processor is further configured to:
determine a diabetes risk level by comparing a trend of a change in glycemic variability indices between two time points with a predetermined threshold value; and
based on determining the diabetes risk level, vary at least one of a thickness, a color, and a type of the second visualization object connecting the positions of the glycemic variability indices at the two time points.

7. The health management apparatus of claim 1, wherein the processor is further configured to:
detect a rising interval, in which the glycemic variability index continuously rises, or a falling interval in which the glycemic variability index continuously falls; and
generate a third visualization object indicating a rising trend of the rising interval or a falling trend of the falling interval.

8. The health management apparatus of claim 7, wherein the processor is further configured to:
based on a size of the rising interval or the falling interval, determine a size of the third visualization object.

9. The health management apparatus of claim 1, wherein the processor is further configured to:
in response to the glycemic variability index being less than a first threshold value, determine that the glycemic variability index decreases;
in response to the glycemic variability index being less than or equal to the first threshold value and being greater than or equal to a second threshold value, determine that the glycemic variability index is maintained;
in response to the glycemic variability index exceeding the second threshold value, determine that the glycemic variability index increases; and
generate a fourth visualization object showing that the glycemic variability index increases, is maintained, or decreases.

10. The health management apparatus of claim 1, wherein the processor is further configured to:
control the display to move a time axis of the trend graph, enlarge a predetermined interval, and select a predetermined time based on a user input received via the user interface.

11. The health management apparatus of claim 1, wherein the processor is further configured to:
predict a diabetes risk level by comparing a slope of a change in the glycemic variability index with a predetermined threshold value; and
control the display to display guide information according to the diabetes risk level.

12. The health management apparatus of claim 1, wherein the processor is further configured to:
predict a diabetes complication risk level by comparing an amplitude of the glycemic variability index during a predetermined period of time with a predetermined threshold value; and
control the display to display guide information according to the diabetes complication risk level.

13. The health management apparatus of claim 1, wherein the processor is further configured to:
control the display to display at least one of the blood glucose variation, the glycemic variability index, and visualization reference information.

14. A health management method, comprising:
obtaining a spectrum from an object;
predicting a blood glucose variation based on the spectrum;
calculating a glycemic variability index based on the blood glucose variation;
generating a trend graph showing a change in the glycemic variability index; and
displaying the trend graph via a user interface.

15. The health management method of claim 14, wherein the trend graph comprises at least one of a first visualization object visually representing positions of glycemic variability indices at predefined times, and a second visualization object connecting the positions of the glycemic variability indices at the predefined times.

16. The health management method of claim 15, further comprising:
determining whether glycemic variability indices between two time points show a rising trend or a falling trend; and
based on determining whether the glycemic variability indices between the two time points show a rising trend or a falling trend, varying at least one of a thickness, a color, and a type of the second visualization object connecting the positions of the glycemic variability indices at the two time points.

17. The health management method of claim 15, further comprising:
determining a diabetes risk level by comparing a trend of a change in glycemic variability indices between two time points with a predetermined threshold value; and
based on determining the diabetes risk level, varying at least one of a thickness, a color, and a type of the second visualization object connecting the positions of the glycemic variability indices at the two time points.

18. The health management method of claim 14, further comprising:
detecting a rising interval, in which the glycemic variability index continuously rises, or a falling interval in which the glycemic variability index continuously falls; and
generating a third visualization object indicating a rising trend of the rising interval or a falling trend of the falling interval.

19. The health management method of claim 14, further comprising:
in response to the glycemic variability index being less than a first threshold value, determining that the glycemic variability index decreases;
in response to the glycemic variability index being less than or equal to the first threshold value and being greater than or equal to a second threshold value, determining that the glycemic variability index is maintained;
in response to the glycemic variability index exceeding the second threshold value, determining that the glycemic variability index increases; and
generating a fourth visualization object showing that the glycemic variability index increases, is maintained, or decreases.

20. The health management method of claim 14, further comprising:
predicting a diabetes risk level by comparing a slope of a change in the glycemic variability index with a predetermined threshold value; and
displaying guide information according to the diabetes risk level.

21. The health management method of claim 14, further comprising:
predicting a diabetes complication risk level by comparing an amplitude of the glycemic variability index during a predetermined period of time with a predetermined threshold value; and
displaying guide information according to the diabetes complication risk level.
